(19) United States
(12) Patent Application Publication
(10) Pub. No.: US 2011/0202089 A1
(43) Pub. Date: Aug. 18, 2011

(54) COMPRESSION HEMOSTASIS DEVICE

(76) Inventor: Dong Sun, Xicheng District (CN)

(21) Appl. No.: 13/119,644

(22) PCT Filed: Jul. 2, 2010
(86) PCT No.: PCT/CN10/74923

§ 371 Date: Mar. 17, 2011

(30) Foreign Application Priority Data
Sep. 30, 2009 (CN) 200920246116.8

(51) Int. Cl. A61B 17/00

(52) U.S. Cl. 606/201

(57) ABSTRACT

The characteristics of the utility hemostasis model is that it includes a buckle plate, guide column, color frame, color cap, T-type platen, elastomer, and turn cap; the described buckle plate is composed of buckle plate body and buckle plate wing, the buckle plate body is a tubular structure with internal thread, and is connected to the guide column by a bolt. The guide column is mounted on the outside of color frame, color cap and T-type platen are inserted from the top to bottom of the color frame and fixed-together. The elastomer is also installed between the described color frame and the guide column. The turn cap which is a cyclic structure is disposed at the top of the guide column, the color cap may be extruded from guide column and turn cap. The structural design of this utility model is reasonable, medical staff can clearly read see the pressure under the rotating state to control hemostasis intensity according to the conditions of the patients; the structure of this utility model is stable and durable. The buckle plate wing is arc structure that meets the physiological curve of the human wrist, and can increase patients wearing comfort during use.

### FIELD OF THE INVENTION

The utility model is a newly developed hemostasis device with good potential for general use. Specifically, a kind of compression hemostasis device for the puncture site of the body to maintain hemostatic equilibrium.

### BACKGROUND OF THE INVENTION

Hemostasis is the maintenance of internal blood levels through the cessation of bleeding. Complex as much as it is critical, the post operative measures that are used to ensure its fluency could define the short term survival and long term well being of the patient. The consequences of disrupted hemostasis after surgical procedures can range from requiring several days of rehabilitation in minor cases to localized ischemic necrosis and possible limb amputation in more serious scenarios. Currently there a variety of different methods that is used routinely in clinical practice. These include interventional therapies via femoral artery, radial artery and vein puncture. The most common method is the compression hemostasis device. This particular piece of apparatus comprises a compression belt with an airbag, in which when airbag is inflated presses the bleeding site and is fastened with a belt holding it in place at the site. There are many problems about such hemostatic devices, such as airbag leakage resulting in volume shrinkage of the airbag. This will in turn lead to pressure reduction at the site which will limit its effectiveness in preventing blood loss. Furthermore the constant need to re-inflate, re-fasten the belt, the need for skilled medical staff to monitor its continuation makes this not only a risky but also inconvenient choice. Due to these problematic technicalities radial artery hemostasis devices were developed as alternatives. This particular device comprises a pressure pad and elastic belt, card, pressure regulating nut, pressure cap, elastic element, buckle plate and T-type platen. The general procedure involved in its operation starts by inserting the card below the bulge on the top of T-type platen and then the pressure regulating nut cooperates with external thread on the top of buckle plate in the middle. The pressure cap is installed between the card and the top of the elastic element through the top of the T-type platen, and its bottom is set at the bottom of pressure regulating nut. The bottom of the elastic element is installed on the upper surface of the bottom of T-type platen, the upper surface also contains a buckle plate, the lower surface contains pressure pad, and the bottom of buckle plate contains elastic belt.. The pressure regulating nut is screwed to transfer the pressure to T-type platen to press the bleeding site. The hemostasis device has improved the above-mentioned deficiencies in existing technologies. The drawbacks of this device is that it does not have a pressure indicative system, medical staff can only determine the suitability of the current pressure via the screwing handle and the observation of the transparent buckle plate body. This might increase uncertainty as to which amount of pressure is most needed to ensure hemostasis. This may in turn lead to unintended ineffective hemostasis results. Another problem involved with the radial artery hemostasis devices is that since the T-type is mounted directly on the pressure cap where the spring is. Pressure is put repeatedly on the site and may cause the T-type platen to be easily damaged ,which undoubtedly reduces the durability of the device, inducing more treatment costs as a consequence. Thirdly, the structural design of buckle plate is unreasonable. The buckle plate contains bent areas which are very close to the elastic belt, when patients use it, wrist muscles and skin are easily trapped during elastic belt fixation increasing patient discomfort.

### SUMMARY OF THE INVENTION

The aforementioned shortcomings of previous hemostasis devices require the introduction of alternative models which can address these issues. We propose our new utility model which not only provides safe and effective hemostatic effects but is also durable, comfortable and easy to use

The utility model has the following technical schemes:

This compression hemostasis device includes a pad and fixed belt. In addition the utility model also has a buckle plate, guide column, color frame, color cap, T-type platen, elastomer, and turn cap. The above buckle plate is composed of buckle plate body and buckle plate wing in which the buckle plate body is a tubular structure with an internal thread. This is connected to the guide column by a bolt. The guide column is mounted on the outside of color frame, in which the color cap and T-type platen are inserted from the top and bottom of the color frame and fixed together; the elastomer is also installed between the described color frame and the guide column. The turn cap that is a cyclic structure is located at the top of the guide column, the color cap may be extruded from guide column and turn cap.

For the above-mentioned compression hemostasis device, has a number of prominent and characteristic design features which are summarized as follows
The buckle plate wing possesses an arc structure that meets the physiological curve of the human wrist.
The elastomer is an spring structure responsible for applying pressure to the site.
A protuberance is arranged on the bottom of color cap, T-type platen contains its matching groove.
The protuberance contains the ring pin, the above groove is equipped with ring slots.
The pad is mounted at the bottom of the T-Plate.
The pad is made of transparent silicone.
The turn cap is equipped with pressure indicative sign to indicate the increase or decrease of pressure.
The fixed belt is an elastic structure, and is connected to buckle plate wing.
The seal ring is installed between the top of the color frame and the guide column.
   Advantages of the Utility Model The advantages and beneficial effects of this utility model are: firstly, pressure indicative sign is installed at the top of turn cap of the utility model hemostasis device; medical staff can clearly read about the pressure under the rotating state to control hemostasis intensity according to the conditions of the patients and achieve individualized care. Secondly, color cap and T-platen are held within the guide column by color frame, which move up gradually through the upward pressure with the downward movement of the guide column. Furthermore, this structure is stable, will not be damaged due to repeating pressure decompression which will increase the life of the device. Thirdly, buckle plate wing of the utility model is an arc structure that meets the physiological curve of the human wrist can be accommodated this will increase the patient wearing comfort during use. The above contents have provided an adequate explanation for this utility model. Below will include a detailed description of the utility model with accompanying drawings and specific embodiments.

### BRIEF DESCRIPTION OF THE DRAWINGS

FIG. 1 Structural view of this utility model under pressure-free state;

FIG. 2 Structural view of this utility model under pressure state;

FIG. 3 Top view of this utility model.

### DETAILED DESCRIPTION OF THE INVENTION

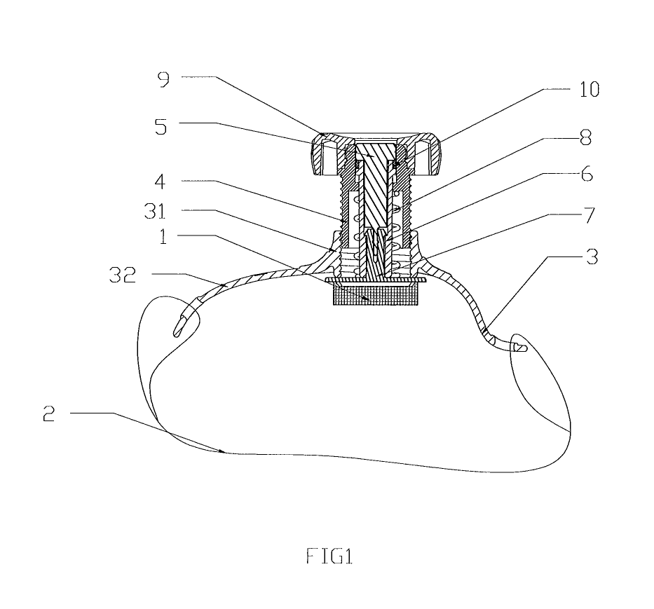

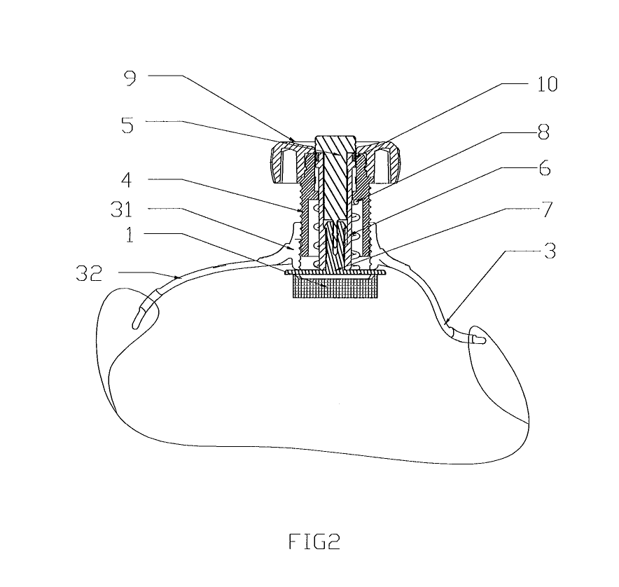

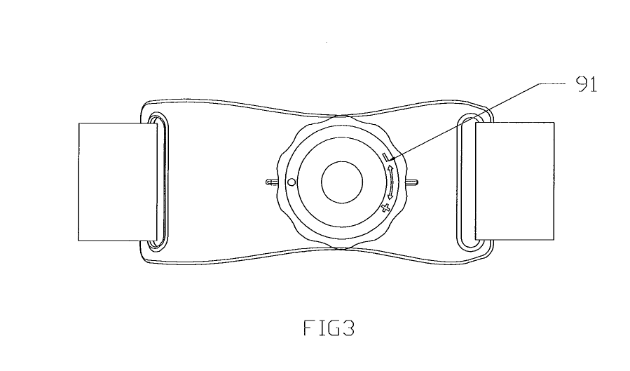

Refer to FIG. 1, FIG. 2 and FIG. 3, the compression hemostasis device, consists of a pad (1) and fixed belt (2), the described hemostasis device also includes a buckle plate (3), guide column (4), color frame (5), color cap (6), T-type platen (7), elastomer (8), and turn cap (9). The buckle plate (3) is composed of buckle plate body (31) and buckle plate wing (32) which is an arc structure that meets the physiological curve of the human wrist. The buckle plate body (31) is a tubular structure with internal thread, and is connected to the guide column (4) by a bolt. The guide column (4) is mounted on the outside of color frame (5), color cap (6) and T-type platen (7) are inserted from the top and bottom of the color frame (5) and fixed-together. Next the elastomer (8) is installed between the described color frame (5) and the guide column (4); in this embodiment, the elastomer (8) is a spring; seal ring (10) is installed between the top of the color frame (5) and the guide column (4).

The turn cap (9) is a cyclic structure and is situated at the top of the guide column (4). The color cap (6) may be extruded from guide column (4) and turn cap (9). The pad (1) is situated at the bottom of the T-type platen (7) and is made of transparent silicone in order to observe patient bleeding.

The bottom of color cap (6) contains the protuberance with the ring pin; T-type platen (7) contains its matching groove that is equipped with ring slots.

Pressure indicative sign 91 is installed on the upper surface of turn cap (9) to indicate whether increasing or decreasing of the pressure is appropriate. In this embodiment, the fixed belt (2) is connected to the buckle plate wing (32), the elastic structure can also be chosen according to specific conditions during use.

The usage and the working principle of this utility model is illustrated with the hemostasis example after radial artery puncture: first, a patient aligns a pad with an bleeding site, and fastens the fixed belt around the wrist; then, medical staff screw the turn cap to the required pressure in accordance with the pressure indicative sign . For the process of screwing the turn cap the guide column and the buckle plate move down along the internal thread, meanwhile a upward pressure from the wrist of the patient transfers to the T-type platen that is connected to the color cap within the color frame and drive color cap to move up. At this time medical staff can re-confirm the pressure state in accordance with the movement state of the color cap. The protuberance of color cap 5 from turn cap is excessive, indicating the pressure is too large. The turn cap needs to be adjusted again according to pressure directive signs.

This utility model can determine the pressure state with the pressure indicative sign on the surface of the turn cap and the motion state of color cap , which is clear and feasible compared with the existing technologies The structural design of the color cap, T-platen and the color frame will not only improve its hemostatic effect, but also has a stable structure which can withstand damage enhancing its durability. The buckle plate wing is arc structure that meets the physiological curve of the human wrist, making it a comfortable choice.

1. A compression hemostasis device, including a pad and fixed belt; its characteristics is that the hemostasis device also includes the buckle plate, guide column, color frame, color cap, T-type platen, elastomer, and turn cap. As previously described the buckle plate is composed of buckle plate body and buckle plate wing, the buckle plate body is a tubular structure with internal thread, and is connected to the guide column by a bolt. The guide column is mounted on the outside of color frame, color cap and T-type platen are inserted from the top and bottom of the color frame and fixed-together; the elastomer is also installed between the described color frame and the guide column; the turn cap which is a cyclic structure is situated at the top of the guide column, the color cap may be extruded from guide column and turn cap.

2. Compression hemostasis device as defined in claim 1, characterized in the buckle plate wing is a arc structure that meets the physiological curve of the human wrist.

3. Compression hemostasis device as defined in claim 1, characterized in that the elastomer is a spring.

4. Compression hemostasis device as defined in claim 3, characterized in that the bottom of the color cap contains the protuberance, T-Plate contains its matching groove.

5. Compression hemostasis device as defined in claim 1, characterized in that the described protuberance contains the ring pin, the above groove is equipped with ring slots.

6. Compression hemostasis device as defined in claim 5, characterized in that the pad is mounted at the bottom of the T-Platen.

7. Compression hemostasis device as defined in claim 6, characterized in that the pad is made of transparent silicone.

8. Compression hemostasis device as defined in claim 6, characterized in that the surface of the turn cap is equipped with pressure indicative signs to indicate the increase or decrease of pressure.

9. Compression hemostasis device as defined in claim 8, characterized in that the fixed belt is gluing structure or elastic structure, and is connected to buckle plate wing.

10. Compression hemostasis device as defined in claim 9, characterized in that seal ring is installed between the top of the color frame and the guide column

11. Compression hemostasis device as defined in claim 2, characterized in that the elastomer is a spring.

12. Compression hemostasis device as defined in claim 2, characterized in that the described protuberance contains the ring pin, the above groove is equipped with ring slots.

13. Compression hemostasis device as defined in claim 4, characterized in that the described protuberance contains the ring pin, the above groove is equipped with ring slots.

14. Compression hemostasis device as defined in claim 7, characterized in that the surface of the turn cap is equipped with pressure indicative signs to indicate the increase or decrease of pressure.
